(19) United States
(12) Patent Application Publication
(10) Pub. No.: US 2018/0250159 A1
(43) Pub. Date: Sep. 6, 2018

(54) Personal Microclimate Systems And Methods

(71) Applicant: Germain Edward DeSeve, III, Philadelphia, PA (US)

(72) Inventor: Germain Edward DeSeve, III, Philadelphia, PA (US)

(21) Appl. No.: 15/912,706

(22) Filed: Mar. 6, 2018

Related U.S. Application Data
(60) Provisional application No. 62/467,312, filed on Mar. 6, 2017.

(51) Int. Cl. A61F 7/00; A41D 13/005

(52) U.S. Cl. CPC A61F 7/007; A61F 2007/0233; A41D 13/0051

(57) ABSTRACT

A personal microclimate control system for sensing cutaneous conditions on a body. The sensors are located on spatially distinct physical locations on a body, adjacent to the cutaneous layer. A controller and connected memory stores the addresses for the sensors. The controller operates the sensors based on their addresses for the purpose of improving thermal comfort. In an embodiment, the controller controls the sensors by executing multiple-input multiple output algorithms. Embodiments are further directed to controlling a plurality of individual electronic effectors as well as to improving system safety, usability and management capabilities.

### CROSS REFERENCE TO RELATED APPLICATIONS

This application claims priority to U.S. Provisional Patent Application No. 62/467,312 filed on Mar. 6, 2017 by the present inventor, and entitled “Personal Microclimate System and Methods”, the contents of which are hereby incorporated by reference in their entirety herein.

### BACKGROUND

Introduction

Under certain conditions, thermal comfort can be maintained independently of core body temperature or the temperature of the ambient environment. These conditions can be created by activating subcutaneous thermoreceptors using electronic effectors such as fans and heat exchangers. This activation can be optimized by using digital controls that are capable of carefully managing variables relating to time, temperature and heat exchange. In order for these controls and effectors to be effective, they must have data from sensors capable of reading cutaneous conditions such as temperature, moisture and air movement.

Sensory Adaptation

Efficient and effective delivery of personal thermal comfort is complicated by a physiological phenomenon known as “sensory adaptation”. Because of sensory adaptation, subcutaneous thermoreceptors in warm blooded animals will stop registering thermal inputs within a matter of seconds. However, sensory adaptation is specific to the area of the body covered by the nerve bundle that include the affected thermoreceptors. It is only possible to achieve truly personalized thermal comfort—a personal microclimate—by overcoming sensory adaptation at multiple spatially distinct locations on a body.

Contemporary Approaches to Thermal Comfort

The most common contemporary prior art approach for actively maintaining thermal comfort is to use air to affect cutaneous temperature. Heat is exchanged with the air via conduction. The treated air is moved to the ambient environment near a subject via convection. The conductance of the treated air's temperature through the subject's skin ultimately reaches their subcutaneous thermoreceptors. This approach is used in typical heating, ventilation and air conditioning (HVAC) systems, such as the Carrier Infinity 20 heat pump. Heated clothing, such as the jackets supplied by Ralph Lauren to the United States team for the 2018 Winter Olympic Games, uses this approach as well, but doesn't move air through convection.

Using ambient air to activate a subject's subcutaneous thermoreceptors is energy intensive because it requires treating a large volume of air relative to the surface area of the subject's skin. In addition, air is a very inefficient medium for conducting temperature due to its specific heat.

The second most common contemporary prior art approach for actively maintaining thermal comfort is to use a liquid medium for the purpose of core body temperature change. Heat is exchanged with the liquid via conduction. The treated liquid is moved across the surface of the subject's body using a means such as pumping through tubing, or simply placed on top of their skin in a sealed unit. The conductance of the treated liquid's temperature through the subject's skin ultimately reaches their subcutaneous thermoreceptors, delivering a sense of thermal comfort, and takes heat from their body's core. This approach is used in typical commercial personal cooling products, such as the combined F.A.S.T. Personal Cooling System and F.A.S.T. Personal Cooling Shirt by RINI.

Using liquid to activate a subject's subcutaneous thermoreceptors requires that a subject be able to support a significant amount of weight, since the amount of liquid required makes the system heavy. Many of these systems are made heavier still by requiring the attachment of air-conditioner type treatment units, such as portable compressors and their related power supplies. The prior art without treatment units can make the subject uncomfortable since they are pre-cooled to a very low temperature prior to use, which chills the subject. Liquid thermal comfort systems are unpractical for many applications since there is the risk that the system could be punctured and release the liquid onto the subject or nearby equipment.

A third approach found in contemporary prior art for actively maintaining thermal comfort uses one or more electronic heat exchangers, typically a thermoelectric module. Each heat exchanger is placed adjacent to the skin and exchanges heat with the skin through conduction. The heat exchange affects the subject's subcutaneous thermoreceptors, delivering a sense of thermal comfort. This approach can be seen in the Wave bracelet from EMBR Labs, which uses a single thermoelectric heat exchanger, and the Dhama Innovations Flowtherm Vest, which uses eight electronic heat exchanger stacks.

Prior art using electronic heat exchangers lacks effective control technologies. These systems run on an “open loop” basis where heat exchange is controlled solely based on current delivery to a heat exchanger, typically a thermoelectric module. An example of this can be found in patent application WO2015054615A1 from EMBR Labs which claims sensors as part of the disclosed bracelet apparatus but does not claim a method for associating those sensors with cutaneous conditions for the purpose of controlling cutaneous temperature. As a result, this approach to prior art lacks the sensor systems and closed-loop control methods necessary for sophisticated management of thermal comfort, such as those that would enable effective avoidance of sensory adaptation. Also, these systems rely heavily on thermoelectric modules and cannot be adapted to use other effectors.

Because of the drawbacks of air, liquid and open-loop electronic heat exchange, prior art cannot effectively control heat exchange to activate thermoreceptors based on specific strategies described in scientific research. The same is true of prior art using passive approaches to maintaining thermal comfort, such as clothing and behavior modification. As such, there is a need for an improved system and control capabilities in order to deliver thermal comfort in novel ways, and increase energy efficiency.

Multiple-Input Multiple Output Control

There are two primary types of closed-loop system control: single-input single-output (SISO) and multiple-input multiple output (MIMO). Because the disclosed apparatus and the embodiments described herein are comprised of a plurality of effectors as well as a plurality of sensors, the closed-loop control methods in the embodiments disclosed herein are all necessarily of the MIMO type. Prior art does not use MIMO control.

In order for an apparatus to effectively manage thermal comfort with a MIMO control algorithm, it is necessary to obtain data on cutaneous conditions, such as temperature. This is necessary because, while a device such as a heat exchanger may be adjacent to the cutaneous layer, it is likely to be enclosed in layers of material, as well as subject to variability in output resulting from interaction with external conditions. As such, it cannot be assumed that device temperature and the temperature reaching subcutaneous thermoreceptors are equivalent. It can also be advantageous to collect other condition data at the cutaneous layer, such as humidity, air speed, or solar heat gain. Prior art does not use cutaneous condition data as input for MIMO control.

In the embodiments disclosed herein, each electronic effector for affecting cutaneous conditions and their related sensors are attached to the apparatus so that they are spatially distinct from the other effector/sensor pairs in the matrix. These physically separated sets of multiple sensor inputs and multiple effector outputs are required to effectively overcome sensory adaptation. Because of their physical separation, the effectors and sensors must have specific addresses within the controller for identifying their unique locations on the body. Prior art does not use addressable effectors or sensors for the purpose of managing thermal comfort using MIMO algorithms.

Applying Comfort Research to Personal Microclimates Using Multiple-Input Multiple Output Control

The concepts underlying the research-driven MIMO comfort control strategy algorithms are found in the five numbered sections immediately below.
1) The “rate of change” algorithm utilizes research from Herbert Hensel and Frithjof Konietzny showing that increasing the rate of temperature change results in a directly related increase in the average impulse frequencies of warm thermoreceptor units on human hairy skin. For example, within a 5 second (s) interval a temperature increase (dT) of 0.5 degrees Celsius (° C.) creates a mean impulse frequency of less than 5 s⁻¹ while dT of 1.5° C. creates impulse frequency of 15 s⁻¹ at the same interval—a difference of more than 300%. Given these results, it is advantageous to operate the heat exchanger at its maximum output in order to achieve the greatest rate of change (dT). However, it is necessary to modulate heat exchange using a closed-loop control strategy so that the exchange is not uncomfortable to the individual using the apparatus.
2) Also based on research by Hensel and Konietzny, the “magnitudes of thermal increments” (ΔT) algorithm reads the cutaneous temperature and a target cutaneous temperature and limits the heat exchange to the difference between those amounts. In an embodiment, applying the result described in the previous paragraph, it can be advantageous to operate the heat exchanger at its maximum output in order to achieve the greatest rate of change (dT). However, when applying this approach, the heat exchange must be modulated using a closed-loop control strategy so that the exchange is not uncomfortable to the individual using the apparatus.
3) The “adapting temperatures” (T_{A}) algorithm, also based on research by Hensel and Konietzny, maintains a static cutaneous temperature at which the temperature change starts. This research demonstrated that increasing the adapting temperature results in a directly related increase in the average impulse frequencies of warm thermoreceptor units on human hairy skin. For example, within a 5 second (s) interval, when warming a single warm fiber from human hairy skin at a constant rate of 1.5° C. s⁻¹ and T_{A} of 32° C. creates an impulse frequency of approximately 15 s⁻¹ while the same rate applied at T_{A} of 37° C. creates an impulse frequency of approximately 35 s⁻¹ at the same interval—a difference of more than 230%. Since thermoreceptor response is greater at higher T_{A}, it is advantageous to maintain a cutaneous temperature that is not at its typical static level.
4) The counterstimulation algorithm works by activating specific thermoreceptor nerve fibers for the purpose of blocking uncomfortable thermal sensations. Human thermoreception takes place through cutaneous myelinated “fast” afferent (Aδ) nerve fibers, primarily non-noxious cold sensing slow-adapting mechanoreceptor fibers (SA fibers) and warmth-sensing Type I and Type II fibers. The activation of Aδ fibers at extreme temperatures inhibits the ability of sensations from the cutaneous unmyelinated “slow” afferent (C) warm-sensing and cold-sensing fibers to reach the cerebrum—a phenomenon described by gate control theory by Ronald Melzack and Patrick Wall. As a result of gate control, the dominant sensation of temperature reaching the cerebrum is the warming or cooling being generated by the apparatus, and not the ambient air—an effect described as “counterstimulation”. Counterstimulation can be thought of as changing typical neurophysiological feedforward outcomes using alternative feedback. Typically, counterstimulation has been used for the purpose of managing sensations relating to pain, such as with Transdermal Electrical Nerve Stimulation (TENS) devices. Disclosed herein is a novel use of counterstimulation which can be applied because sensations relating to temperature and pain both travel via Aδ and C fibers, and so are subject to counterstimulation. This use of counterstimulation is advantageous because it enables thermal comfort without requiring changes to the ambient conditions
5) The algorithm using passive “attention diversion” is based on research findings which “suggest attention plays an important part in the pain relief experienced from counter-stimulation” (Longe et al 2001). Since pain and thermal sensations both travel via the same nerve fibers, it can be assumed that attention diversion can provide relief from uncomfortable temperatures. Given this, when direct mental focus—attention—is diverted from being uncomfortably hot or cold, the resulting experience is indistinguishable from the sensation of experiencing thermal comfort. This can be advantageous because the apparatus requires less energy to power the system when attention is diverted from thermal comfort.

Each of these effects are used in different embodiments described herein, as summarized in the five numbered paragraphs immediately below:
1) To create a rate of change of cutaneous temperature, according to an embodiment, the amount of temperature applied by the heat exchangers is regulated during a specific increment of time.
2) To create magnitudes of thermal increments, according to an embodiment, the total change in temperature (ΔT) during heat exchange is limited to the difference between a cutaneous temperature reading and the desired cutaneous temperature. In the disclosed embodiments, ΔT is limited to a specific range within the boundaries of noxious hot and cold sensations as defined by medical research, which are summarized in the table in FIG. 11C.
3) To create an adapting temperature, according to an embodiment, static cutaneous temperature at the site of a heat exchanger is maintained at a level that is different from a typical static level.
4) To create a counterstimulation effect, according to an embodiment, heat exchange activates Aδ nerve fibers which, in turn, block the C fiber activity which degrades sensations of comfort.
5) To passively divert attention using gate control theory, according to an embodiment, cycles of heat exchange occur with decreasing frequency until there is a focusing of mental activity on the lack of thermal comfort and a user-operable control for resetting the cycles' frequency is activated.

Advantages

Application of the disclosed control methods enable a highly efficient means for delivering thermal comfort since they do not attempt to change ambient environmental conditions, or to change a subject's core temperature. Further, application of these methods are not limited to being located at a specific area of the subject's body, such as their wrist, torso, limbs, or extremities. As a result, the disclosed personal microclimate control system and method can be used to deliver thermal comfort in a variety of innovative ways. The system is also highly flexible: it can applied to products including garments, wearable apparatuses, personal protective equipment and furniture.

Those skilled in the art will appreciate that several additional advantages over prior art related to thermal comfort have been incorporated into the embodiments herein, including: increasing usability; enhancing the manufacturability of the apparatus using printed circuits; making the control enclosure separable from the rest of the apparatus; utilizing a standard cable between the controls and the effector matrix; sealing the apparatus in watertight material to prevent ingress of liquids or debris; and enabling the system to be used with a primary as well as a secondary battery.

### SUMMARY

Embodiments disclosed herein are directed toward sensing cutaneous conditions on a body through operation of addressable sensors. The sensors are managed by a connected controller and memory storage. Each sensor is attached to a unique location on a body. Addresses for the physical locations of the sensors are stored in memory. The controller operates the sensors based on their addresses. In an embodiment, the controller controls the sensors by executing multiple-input multiple output algorithms. Embodiments are further directed to controlling a plurality of individual electronic effectors as well as to improving system safety, usability and management capabilities.

### BRIEF DESCRIPTION OF THE DRAWINGS

The foregoing and other aspects of the invention are best understood from the following detailed description when read in connection with the accompanying drawings. The drawings depict embodiments solely for the purpose of illustration; it should be understood, however, that the disclosure is not limited to the specific instrumentalities disclosed. Included in the drawings are the following Figures:

FIG. 1: Apparatus Drawing—a diagram illustrating the disclosed apparatus, for use as a reference for the detailed description of the disclosed embodiments;

FIGS. 2A-2D: Apparatus Drawings With Effectors—are diagrams illustrating the disclosed apparatus which, in an embodiment, includes effectors, for use as a reference for the detailed description of the disclosed embodiments;

FIGS. 3A-3C: User Control Drawings—are diagrams illustrating embodiments for user control of the disclosed apparatus, for use as a reference for the detailed description of the disclosed embodiments;

FIGS. 4A-4C and FIG. 5: Specification Tables—are tables of information detailing the operation of the disclosed apparatus, for use as a reference for the detailed description of the disclosed embodiments;

FIG. 6: a flowchart illustrating an embodiment where the disclosed apparatus utilizes a “Baseline” MIMO control strategy for the purpose of managing effector activity at a plurality of effectors using closed-loop feedback for the purpose of enabling effective control over the apparatus;

FIG. 7: A flowchart illustrating an embodiment where the disclosed apparatus utilizes a “time element” MIMO control strategy for managing activity at a plurality of effectors during a set period of time for the purpose of efficiently managing the effectors;

FIG. 8 is a flowchart illustrating an embodiment where the disclosed apparatus utilizes a “safety cutoff” MIMO control strategy for management of effectors to ensure non-harmful system operation;

FIGS. 9A-9B are flowcharts illustrating an embodiment where the disclosed apparatus utilizes a “dynamic energy efficient” MIMO control strategy for adjusting power delivery to effectors based on the ambient temperature;

FIGS. 10A-10B are flowcharts illustrating an embodiment where the disclosed apparatus utilizes a “rate of change” MIMO control strategy for management of effectors wherein pre-determined values for temperature change as a function of time at various voltages;

FIGS. 11A-11B are flowcharts illustrating an embodiment where the disclosed apparatus utilizes a “thermal magnitude increment” MIMO control strategy for managing effector activity using input from temperature sensors;

FIG. 11C is a specification table with information detailing the operation of the provided apparatus, for use as a reference for the detailed description of the disclosed embodiments;

FIGS. 12A-12B are flowcharts illustrating an embodiment where the disclosed apparatus utilizes a “adapting temperature” MIMO control strategy for managing a consistently maintained elevated cutaneous temperature at a plurality of effectors;

FIGS. 13A-13B are flowcharts illustrating an embodiment where the disclosed apparatus utilizes a “gate control counterstimulation” MIMO control strategy for activation of Aδ nerve fibers related to subcutaneous thermoreceptors; and

FIGS. 14A-14B are flowcharts illustrating an embodiment where the disclosed apparatus utilizes an “attention diversion” MIMO control strategy for optimizing the efficiency of the apparatus by delaying effector operation if the user's attention is not focused on their thermal comfort.

### DETAILED DESCRIPTION

Apparatus Drawing

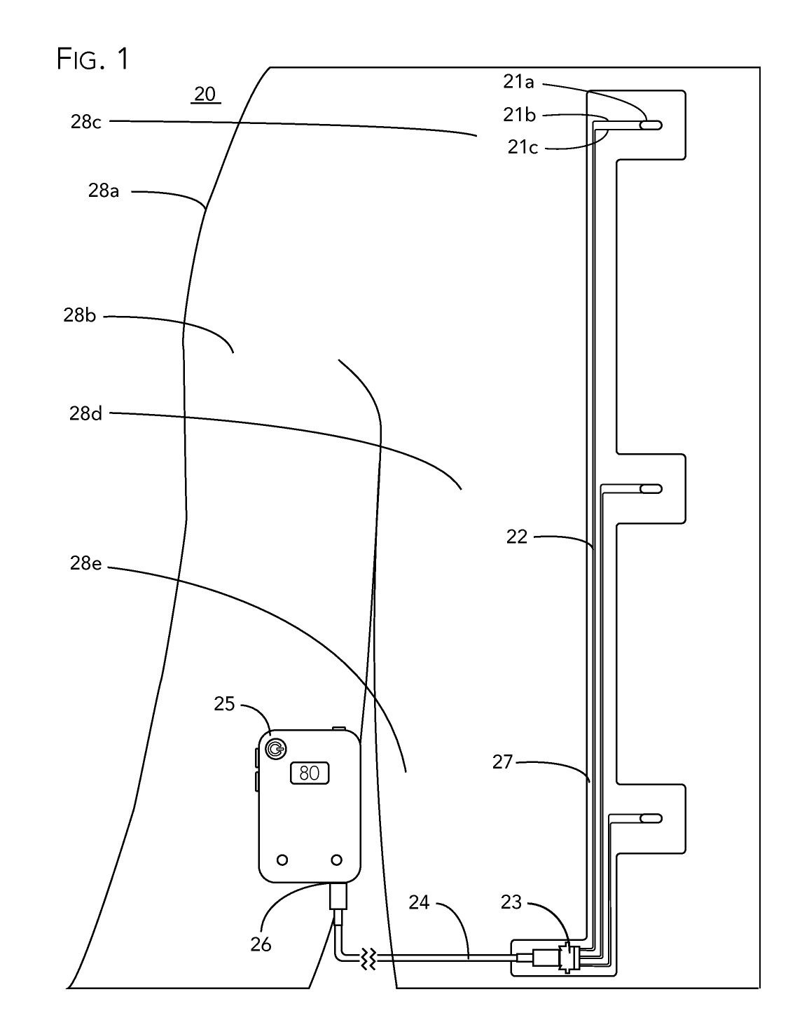

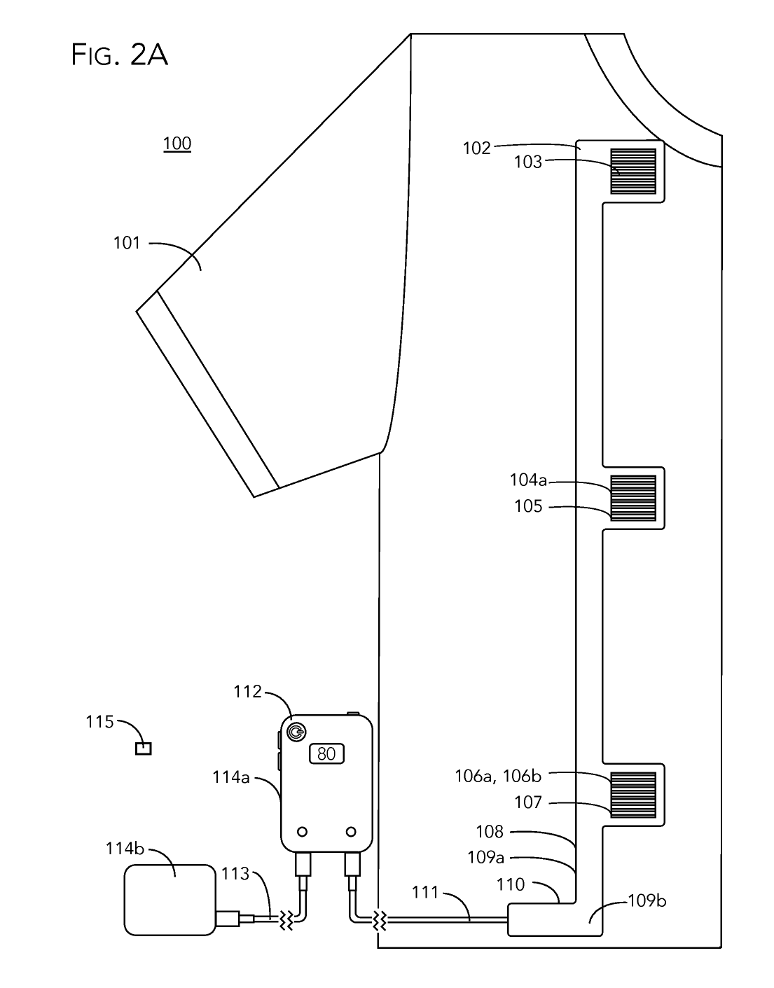

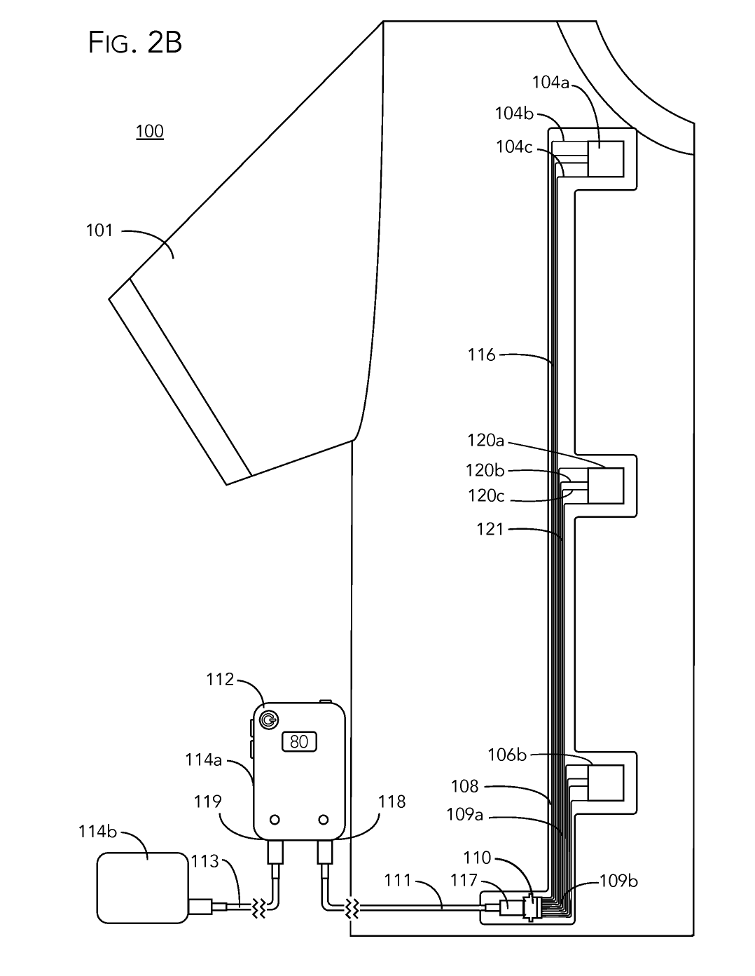

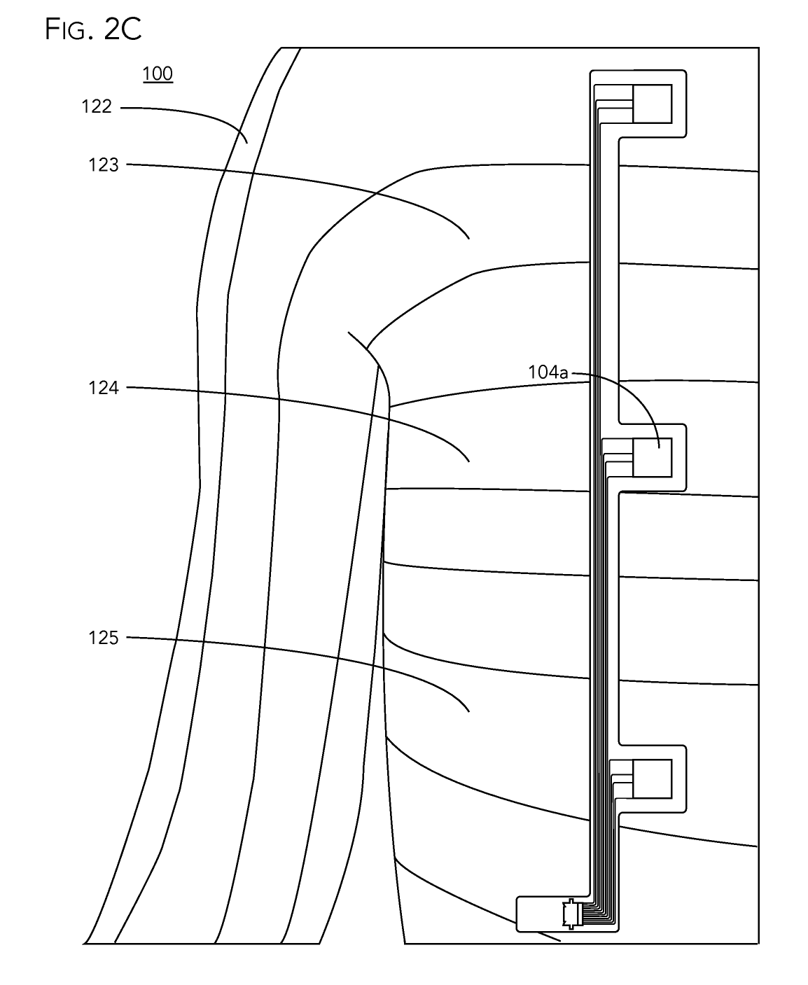

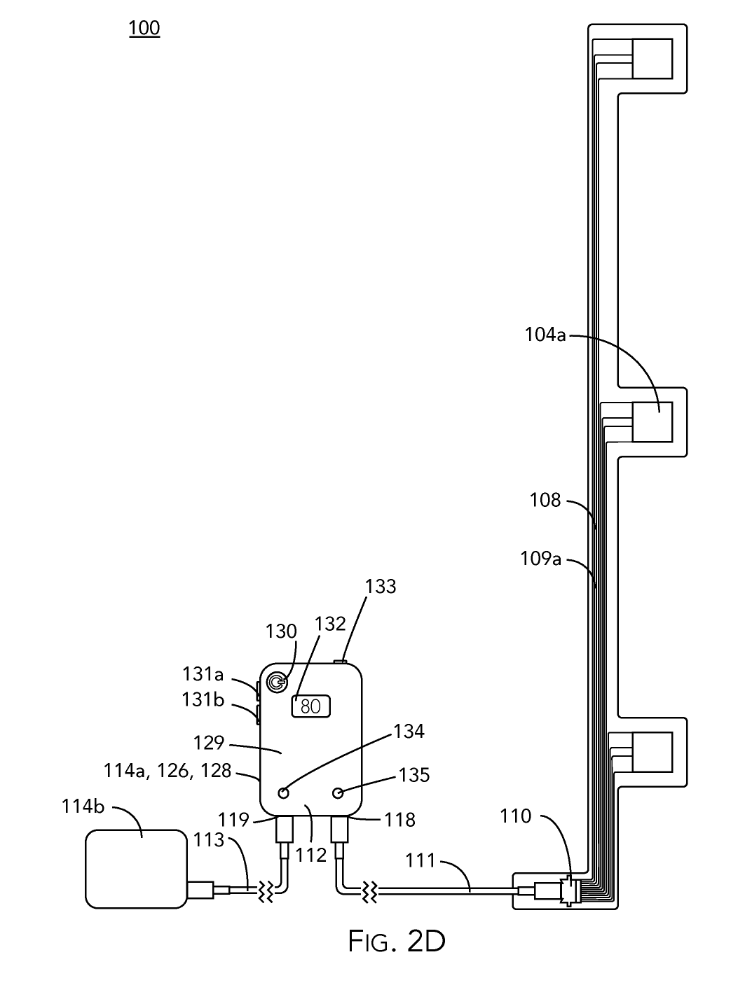

FIG. 1 is an exemplary representation of the disclosed apparatus illustrating an apparatus 20, according to an embodiment. FIGS. 2A-2C are exemplary representations of the disclosed apparatus being utilized in a garment, according to an embodiment. FIG. 2A illustrates an outer-most view of the apparatus 100; FIG. 2B illustrates an interior view of an apparatus 100; FIG. 2C illustrates how the apparatus 100 corresponds to unique physical locations on a body; and FIG. 2D illustrates an interior view of the apparatus 100 with special attention given to the digital control unit controller.

With reference to FIG. 1, in an embodiment, there are three cutaneous condition sensors 21a, with leads for power 21b and ground 21c. In an embodiment the cutaneous condition sensors 21a are temperature sensors for sensing temperature and humidity, such as a DHT-11 type. In an alternative embodiment, the cutaneous condition sensors 21a could be of a thermistor or thermocouple type, such as an NTC thermistor type.

In an embodiment, the cutaneous condition sensors 21a have leads 21b and 21c with wired connections 22 to the matrix connector 23. In an embodiment, the wired connections 22 are of an insulated wire type and the matrix connector 23 is of a universal serial bus (USB) type. The matrix connector 23 is connected to the matrix-controller cable 24. In an embodiment, the matrix-controller cable 24 is of a USB type. The matrix-controller cable 24 connects to the controller 25 using the matrix port 26. In an embodiment, the matrix port 26 is of USB type. The controller 25 is connected to electronic memory storage (not shown) capable of storing executable code. The controller 25 has addressable ports on its central processing unit (not shown) for connecting to and controlling the power leads 21b of the cutaneous condition sensors 21a and a ground for connecting to the ground leads 21c of the condition sensors 21a. The controller 25 is of a type that can execute commands using code.

The cutaneous condition sensors 21a, in an embodiment, are physically attached in place by an adhesive film 27 such that the cutaneous condition sensors 21a face toward, and are immediately adjacent to, a body 28a. The adhesive film 27 also serves to hold the cutaneous condition sensors 21a adjacent to the body 28a and, specifically, the cutaneous layer 28b of the body 28a. The adhesive film 27 further holds the cutaneous condition sensors 21a in unique physical locations a body 28a: the upper torso 28c, middle torso 28d and lower torso 28e.

Apparatus Drawings with Effectors

With reference to FIGS. 2A-2C, in an embodiment, an adhesive film layer forming a channel cover 102 holds components securely in place at physically unique locations on a garment 101 and protects them from damage. The channel cover 102 also prevents the components from unsafely catching on external objects and/or body parts, etc. The adhesive film can be a commercially-available thin polyurethane (TPU) type, such as Bemis Sewfree Exoflex with Ash pigment. Use of TPU for this purpose is advantageous because it is waterproof and returns to its original shape after being stretched—which enables the effectors 104a to remain consistently adjacent to a body's cutaneous layer and subcutaneous thermoreceptors (not shown) even as the body holds various postures and performs movements. Use of a pigmented TPU cover is advantageous during manufacturing because it eliminates the resources required to purchase fabric, cut it and laminate it to a separate piece of TPU film.

In an embodiment, a plurality of cutaneous condition sensors 120a are associated with the plurality of effectors 104a. In an embodiment, the cutaneous condition sensors 120a are placed adjacent to the garment-facing surface of the effectors 104a between the thermally conductive adhesive 106b and the base layer of waterproof adhesive 108 such that they are able to read the conditions on the cutaneous layer of a body (not shown). In an embodiment, the cutaneous condition sensors 110a are a commercially available type, such as a 10K Ohm thermistor. The cutaneous condition sensors 120a have associated leads for power 120b and ground 120c. The use of cutaneous condition sensors is advantageous because it enables management of closed-loop control of the apparatus via the software application and related algorithms. Specifically, an embodiment enables closed-loop multi-input multi-output (“MIMO”) control over the apparatus. In an alternative embodiment, a plurality of cutaneous condition sensors can be arranged such that they are able to obtain readings from the body's cutaneous layer by placing the sensors below the surface of the garment 101 facing the skin and adjacent to the skin (not shown). In another alternative embodiment, cutaneous condition sensors can be used in concert with heat exchanger temperature sensors (not shown).

In an embodiment, the cutaneous condition sensors' wires 120b and 120c have trace connections 121 to the matrix connector 110. In an embodiment, the trace connections 121 are conductive traces printed using a stretchable electrically conductive ink solution capable of being printed on the base layer of waterproof adhesive 108, such as the DuPont PE872 conductor and PE772 encapsulant.

A plurality of heat sinks 103, in an embodiment, are attached to the channel cover 102 so they are associated with the respective ones of a plurality of effectors 104a. The channel cover 102 is cut 105 to allow exposure of the outward facing surfaces of the effectors 104a through the channel cover 102. In an embodiment, the heat sinks 103 can be an aluminum type. In an embodiment, the effectors 104a can be a commercially-available heat exchanger of a thermoelectric module (TEM) type, such as a TEC1-04902. In an alternative embodiment, the effectors 104a could be a thermoelectric heat exchange material applied to a substrate by means such as screen printing. In an alternative embodiment, the effectors 104a can be a commercially available vertical draft fan, such as a Sunon model UF383-100, or a horizontal blower fan type, such as a Sunon model B0503AFB2-8(MS). In an alternative embodiment, the effectors 104a can be piezoelectric air movers such as a SynJet XFlow 30. In another embodiment, the heat exchange from the effectors 104a could take place using a liquid, or a phase-change material.

In an embodiment, the effectors 104a are placed so a cooling heat exchange effect will be directed toward the garment 101 and the body. This effect creates waste heat on the side of the effectors 104a facing away from the garment 101. In an alternative embodiment, the polarity of the power being supplied to thermoelectric module (TEM) type effectors 104a can reversed via a user-operable mechanism such as a dual-pole dual-throw (DPDT) switch (not shown) so that the TEM type effectors 104a provide heat toward the garment 101.

In an embodiment, the heat sinks 103 are a type with a cavity facing the effectors 104a such that the heat sinks 103 and effectors 104a can be paired without any excess space between their surfaces other than that needed for any adhesives. This arrangement is advantageous because it reduces the overall height of the combined heat sinks 103 and effectors 104a apparatus and increases ruggedness because the heatsinks 103 can form a protective physical layer for the effectors 104a.

In an embodiment, the plurality of heat sinks 103 is of a surface area large enough that they are able to perform natural convection to fully eject heat removed by the effectors 104a. This is advantageous during use because no moving parts are required for heat ejection—such parts would be prone to breakage, fouling or other issues. The arrangement is further advantageous because it improves manufacturability by eliminating heat ejection components such as fans and the resources required to build them into the apparatus. In an alternative embodiment, the apparatus uses a plurality of devices for moving air over the surface of said plurality of heat sinks 103, such as electronic Pulse-Width-Modulation (PWM) or non-PWM fan or blower type. In an alternative embodiment, the apparatus ejects heat with a plurality of electronic PWM or non-PWM solid-state air movers such as ionic wind generator type.

In an embodiment, an adhesive 106a may be used to attach the plurality of heat sinks 103 to the plurality of effectors 104a. In an embodiment, the adhesive may be a thermally conductive type that holds the components together while conducting temperature between them. In an embodiment, the adhesive 106a covers the surface of the top of the effectors 104a. In an alternative embodiment, the adhesive may cover both the sides and the surface of the top of the effectors 104a.

In an embodiment, an adhesive 107 may be used to attach the heat sinks 103 to the outward facing surface of the channel cover 102 using the area adjacent to the cavities of the heat sinks 103. This adhesive may be of a waterproof type, such as silicone. This is advantageous because it provides a layer of sealing against ingress by water or debris.

In an embodiment, the assembly of the channel cover 102, the heat sinks 103, effectors 104a, the effector-heat sink surface adhesive 106a and the heat sink-channel cover adhesive 107 creates a fully sealed bond that is capable of holding said components securely together and in place. This is advantageous because it provides a sealed barrier against ingress by any water or debris that could inhibit safe operation of the apparatus.

In an embodiment, the channel cover 102 may be bonded to a matching base layer of waterproof adhesive 108, such as thin polyurethane film (TPU) adhesive that is heat-activated and pressure-activated, and bonded to the garment 101, with the components placed between the two adhesive layers 102 and 108. The bonded adhesives 102 and 108 create a waterproof matrix 109a for associating the heating elements 104a to the subcutaneous thermoreceptors of the wearer's body and holding said elements securely in place. The bonded effector matrix 109a also secures the matrix connector 110 and matrix-controller cable 111 in place so as to ensure that these components do not disconnect, while also providing strain relief for the matrix-controller cable 111. In an embodiment, the effector matrix 109a contains an angled section 109b before being joined to the matrix connector 110. This is advantageous because it positions the matrix connector 110 so that the matrix-controller cable 111 can be aligned correctly for the optimal attachment to the controller 112.

In an embodiment, the controller 112 is of a digital type that can execute commands using code (a “digital control unit” or “DCU”). The controller 112 is connected to electronic memory storage (not shown) capable of storing executable code. The controller 112 has addressable ports on its central processing unit (not shown) for connecting to and controlling the power leads 120b of the cutaneous condition sensors 120a and the effector power leads (see FIG. 2B 104b) as well as a ground for connecting to the ground leads 120c of the condition sensors 120a and the effector ground leads (see FIG. 2B 104c).

In an embodiment, a molded waterproof cover 115, such as a silicone type, for the matrix-controller cable 111 seals the cable end used for connecting to the DCU 112 to prevent ingress of debris and water when the apparatus is not in use and disconnected from the DCU 112. This approach to completely sealing and waterproofing the effector matrix 109a is advantageous because the apparatus can be cleaned using a typical commercially-available washing machine. In an alternative embodiment, a molded waterproof cover can cover the matrix connector 110.

In an embodiment, an adhesive 106b, may be used to securely hold the garment-facing surface of each effector 104a to the adhesive layer 108. In an embodiment, adhesive is a thermally conductive type that holds the components together while conducting temperature between them.

In an embodiment, the effector matrix 109a is connected via the matrix connector 110 to a DCU 112 using the matrix-controller cable 111. The DCU 112 is powered by an internal rechargeable primary battery 114a such as a lithium-polymer type.

In an embodiment, the DCU 112 is connected to a secondary power supply 114b via a cable 113. In an embodiment, the secondary power supply is a commercially-available type, such as a power bank with a lithium-polymer rechargeable battery and Universal Serial Bus (USB) connections for power output and charging. Use of a commercially-available secondary power supply is advantageous because it provides a simple, affordable and scalable method for increasing the amount of time the apparatus can operate.

In an embodiment, the matrix-controller cable 111 and the secondary power supply cable 113 are a standard commercially-available type such as a USB 2.0 A male to Micro USB B male cable. The use of standard cables is advantageous in manufacturing because they are commercially available in multiple typical lengths. As a result, a typical length can easily be used to meet the needs of a specific application, as opposed to requiring a completely custom cable assembly for every application. This approach lowers manufacturing costs for connecting said components by reducing the amount of resources required during stages including design, tooling and assembly.

According to an embodiment, the effector 104a is comprised of a heat exchanger stack (“HE stack”) which is designed so that heat can be effectively ejected into the external environment. The HE stack can be comprised of the following components, which move heat from the garment 101 to the external environment while holding the stack together and in place:
1) the base layer of waterproof adhesive 108;
2) the thermally conductive adhesive 106b for securing the effectors 104a to the base layer of waterproof adhesive 108;
3) the effectors 104a;
4) the heat exchanger-heat sink surface adhesive 106a; and
5) the heat sinks 103.

The thermally conductive adhesive 106b for attaching the garment 101 and the base layer of waterproof adhesive 108 to the effectors 104a holds the components together while conducting temperature between them. In an embodiment, when electric current is passed through the effectors 104a of a thermoelectric module type, the effectors 104a exchange heat by moving it from one side of the heat exchangers' surface to the other side.

The aluminum in the heat sinks 103 ejects heat away from the effectors 104a by conducting it and spreading it over the surface area of the heat sink 103, which is significantly larger than the surface area of the effectors 104a in an embodiment. The surface area provided by the heat sink 103 promotes natural convective heat exchange between the effectors 104a and the external environment. In an alternative embodiment, the heat sinks 103 can have a plurality of related operating condition sensors (not shown) connected to the controller (see FIG. 1D 128) for the purpose of providing a readings at their surface. This alternative embodiment would be advantageous because it can provide an additional means of ensuring safe system operation by, for example, providing back-up sensing of over-temperature conditions of any of the plurality of effectors 104a.

With reference to FIG. 2B, in an embodiment, the plurality of effectors 104a have associated wires for power 104b and ground 104c. The heat exchanger wires 104b and 104c have connections 116 to the matrix connector 110. In an embodiment, the connections 116 are traces printed on the base layer of waterproof adhesive 108 with a stretchable electrically conductive ink solution, such as the DuPont PE872 conductor and PE772 encapsulant. This approach is advantageous because stretchable conductive ink traces will stretch along with the body while conforming to it, as opposed to typical wires, which will limit range of movement during stretching and can flex away from the body because of their rigidity. Printing traces can also be done in a manner that standardizes the connection points of the matrix 109a arrangement so the points can accommodate multiple heat exchanger types (such as printed resistive heating elements, resistive heating element wire, foil resistive heating elements, thermoelectric elements with ceramic components, printed thermoelectric elements and thin-film thermoelectric elements) without requiring a change in the printed sections of the effector matrix 109a. Such arrangements with various heating elements types can be considered alternative embodiments.

In an embodiment, the matrix connector 110 is a standard commercially-available type, such as a USB Type C female. The use of USB Type C connections is advantageous because it is compact but provides up to twenty-four (24) separate positions for connecting electronic components. USB is also an international standard for connecting electronic components via a bus for supplying power and data.

In an embodiment, the effector matrix 109a is connected to the DCU 112 by connecting one end of the matrix-controller cable 111 to the matrix connector 110 and the other end of the matrix-controller cable 111 to the DCU 112. In an embodiment, the effector matrix 109a and matrix-controller cable 111 can be fully disconnected from the DCU 112 by removing the end of the matrix-controller cable 111 from the matrix port 118. This is advantageous because it allows the garment 101 and effector matrix 109a to be washed. It also allows the DCU 112 to be removed for recharging the primary battery 114a. This is advantageous because the garment 101 does not need to be taken off of the body during charging of the primary battery 114a.

In an embodiment, the secondary battery 114b and power cable 113 can be disconnected from the DCU 112 via the power port 119. Once this has been done, the power port 119 can be used for recharging the primary battery 114a.

In an embodiment, a length of the matrix-controller cable 111 and its connection overmold 117 is sealed between the channel cover 102 and the base layer of waterproof adhesive 108 and bonded to the garment 101 in order to provide strain relief and maintain contact between the cable termination and the matrix connector 110. In an alternative embodiment, the matrix connector alone would be sealed between the channel cover 102 and the base layer of waterproof adhesive 108 and bonded to the garment 101.

With reference to FIG. 2C, in an embodiment, the effectors 104a are used to address three spatially distinct dermatomes 122 as follows, from top to bottom of the effectors 104a: dermatome C5 123; dermatome T3 124; and dermatome T6 125. The surface area of each effector 104a is small enough to address a specific dermatome 122. The physical locations of the dermatomes 122 are advantageous because they are non-adjacent, which minimizes possible negative effects from spatial summation. In alternative embodiments, the method of spatial distinction and/or the location and quantity of dermatomes can be adjusted to suit the particular purpose of an apparatus.

With reference to FIG. 2D, in an embodiment, the digital control unit controller (DCU) 112 is comprised of the electronics 126, primary battery 114a and central processing unit (CPU) 128 housed in an enclosure 129. In this embodiment, the DCU 112 has an enclosure 129 of a molded plastic type. Those skilled in the art will appreciate that the DCU 112 in this embodiment, as well as the function of and arrangement of its related components, is only one of many possible solutions for controlling and powering the apparatus. In an embodiment, touch-sensitive controls (see FIG. 2A) can be mounted on the surface of the garment or the controls can be comprised of a software application running on a smart phone with a wireless connection to components associated with the effector matrix 109a (see FIGS. 2B-2C). In an embodiment, the primary battery 114a has an integrated temperature sensor (not shown) such as an NTC thermistor.

In an embodiment, the DCU 112 can be connected via the matrix port 118 to the effector matrix connector 110 using the matrix-controller cable 111. In an embodiment, the matrix port 118 is a standard commercially-available connector, such as a USB Type C female type.

In an embodiment, the DCU 112 can be connected via a power port 119 to a secondary battery 114b using a power cable 113. In an embodiment, the power port 119 is a standard commercially-available connector, such as a Micro USB B female type. The power port 119 can also be used for recharging the primary battery 114a. In an embodiment, charging would be done via the power cable 113 and a wall pack adapter (not shown) for connecting to a standard electrical outlet. In an alternative embodiment, charging of the primary battery 114a can be done using an induction charging system type.

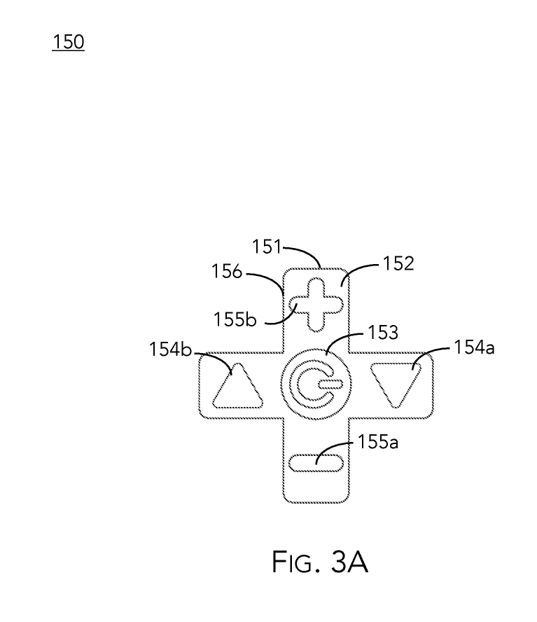

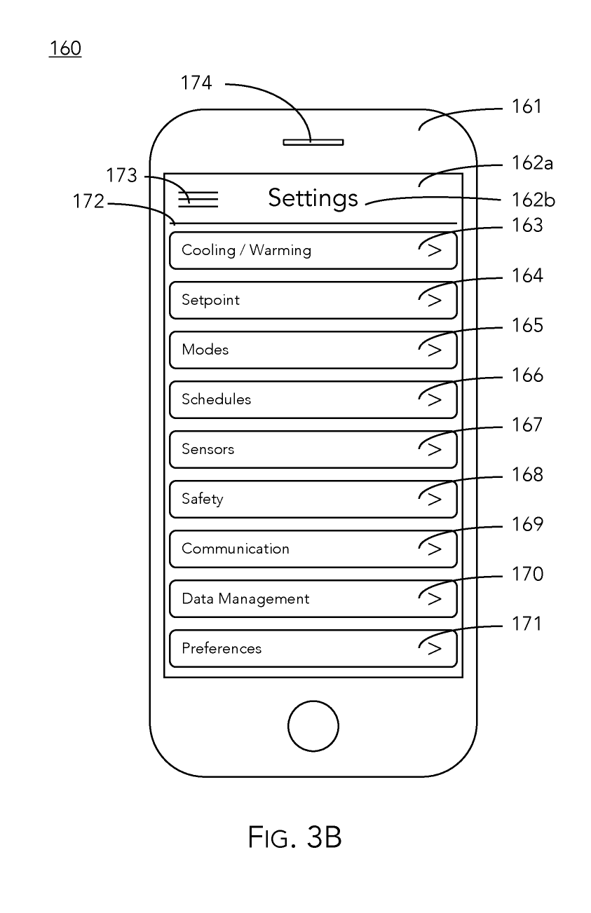

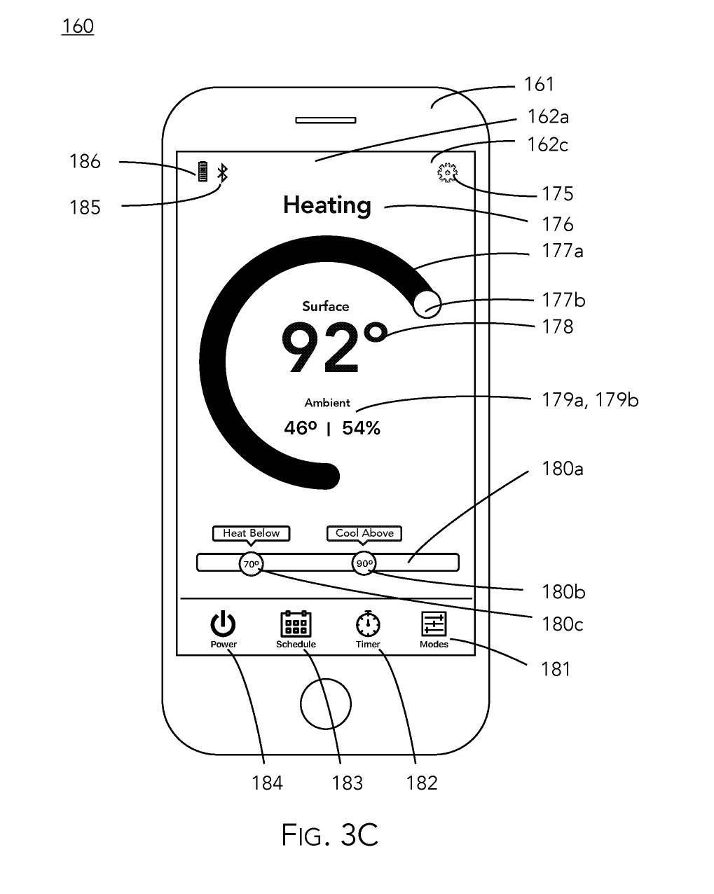

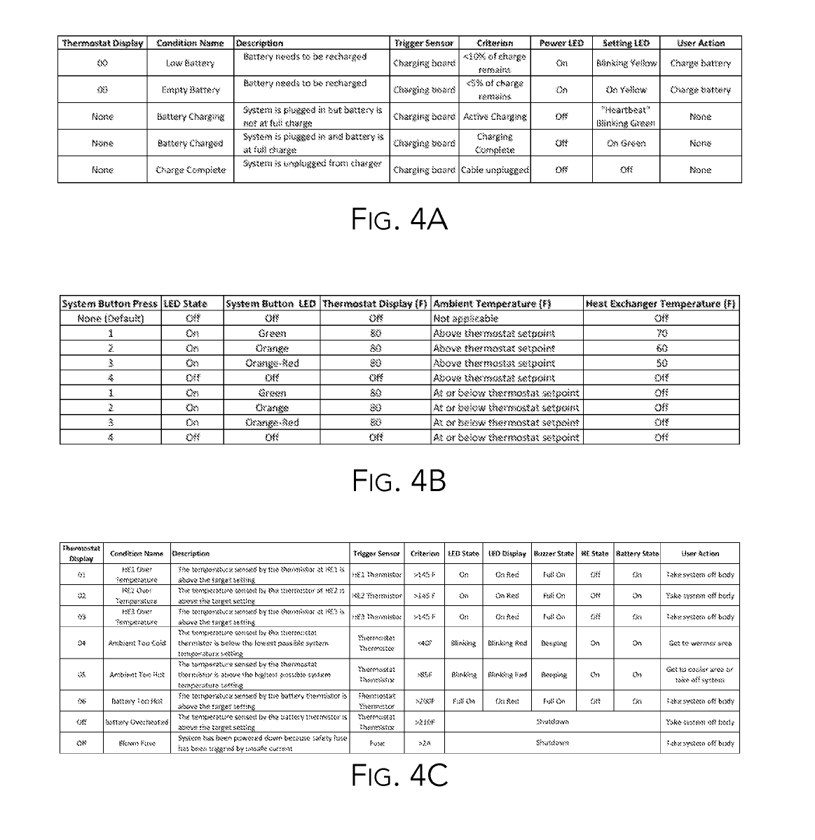

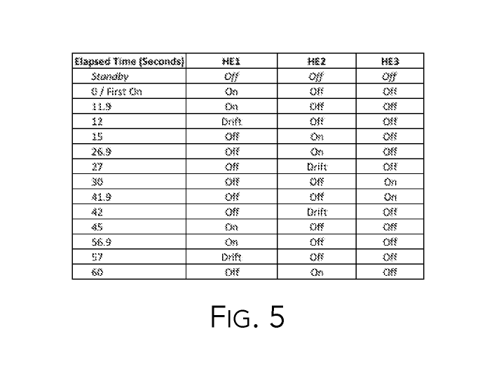

In an embodiment, a user-operable system control 130 connected to the controller 128 adjusts settings. In this embodiment, the system control 130 is a button type illuminated by a light emitting diode (LED) and capable of turning the apparatus on, putting the apparatus into a low-power standby state and changing the output levels of effectors 104a. In an embodiment, the system control 130 also provides display information through colors displayed using the LED to indicate charging state and system errors. See the table in FIG. 4B for additional details on the system control 130 button LED display. In an alternative embodiment, the controller 128 is associated with an integrated wireless device (not shown), such as a Bluetooth type, for wireless system control via user-operable controls physically removed from the DCU 112, such as those in the software application 160 in FIG. 3B and FIG. 3C.

In an embodiment, user-operable controls 131a and 131b enable adjustments to the thermostat setpoint. Controls 131a and 131b are button type connected to momentary switch type contacts connected to the controller 128. Button 131a increases the thermostat setpoint and button 131b decreases the thermostat setpoint. In an alternative embodiment, system controls are contained in a wired configuration physically removed from the DCU 112, such the surface-mounted control panel 150 type shown in FIG. 3A.

In an embodiment, a display 132 shows the user's chosen thermostat setpoint and other information related to operation of the apparatus, such as safety information. In this embodiment, the display is an LCD type with backlighting. See the tables in FIG. 4A and FIG. 4C for additional details on the LCD display, in an embodiment, where it the LCD display is referenced as being of a Thermostat Display type.

In an embodiment, a user-operable control 133 enables restarting of the controller 128 and clearing specific parameters. In this embodiment, the control 133 is a button type.

In an embodiment, there is an opening in the enclosure for a surface-mounted temperature sensor 134 connected to the controller 128 for the purpose of obtaining readings of the ambient temperature for use by the thermostat control.

In an embodiment, there is an opening in the enclosure 135 for allowing audible alerts to be heard by the user. In an embodiment, audible alerts would be generated by a buzzer connected to the controller 128 such as a piezoelectric transducer type.

User Control Drawings

With references to FIG. 3A, in an embodiment, the apparatus 100 is controlled using a surface-mounted control panel 150 connected to the controller (see FIG. 2D, reference 128) with user-operable controls such as a membrane type with momentary switch buttons. The surface-mounted control panel 150 is printed on a flexible substrate 151 and incorporates a circuit board (not shown) such as a flexible printed type. The top layer 152 of the surface-mounted control panel 150 contains the graphical user interface. In an embodiment, these include a system control button 153 capable of turning the apparatus on, putting the apparatus into a low-power standby state and adjusting output of the effectors 104a. In an embodiment, user-operable controls enable adjustments to the thermostat setpoint by decreasing 154a or increasing 154b the setpoint temperature; and heat exchange by decreasing 155b or increasing 155a power to said heat exchangers. The surface-mounted control panel 150 is affixed 156 to the base product using a means such as an adhesive. In an embodiment, the surface-mounted control panel 150 is connected via wires (not shown) to the DCU 112. In an embodiment, the surface-mounted control panel 150 is connected to a wireless device (not shown) for communicating with and controlling settings on the DCU 112. In an embodiment, changes made to the apparatus settings on the surface-mounted control panel 150 are immediately reflected in any other controls, such as a digital control unit type user interface or a software type graphical user interface.

With reference to FIG. 3B and FIG. 3C, in an embodiment, the apparatus 100 is controlled using software 160, such as a smart phone 161 app 162a.

With reference to FIG. 3B, in an embodiment, the software can be comprised of the following 10 numbered elements, as disclosed in the Settings interface 162b of app 162a:
1) Heat exchanger management 163 for: changing the type of heat exchange between warming and cooling; and increasing or decreasing the desired amount of heat exchange;
2) Increasing or decreasing thermostat setpoint 164;
3) Changing between control strategies stored in the app 162a, herein called “Modes” 165. Modes 165 can include, for example: pre-cooling before physical activity; warm-up before physical activity; cool-down after physical activity; settings specific to indoor or outdoor operation; location-specific settings; and user-configurable custom operation based on settings stored in memory.
4) Creating and adjusting “Schedules” 166, herein defined as settings or Modes assigned to operate during specific temporal periods defined by the user, such as hours during days of the week.
5) Associating the app 162a with sensors 167 for obtaining data for use by the apparatus 100 such as wireless and wired analog- or digital-type sensors on said apparatus or spatially removed from it. Sensors can include those for: temperature, such as that from the cutaneous layer, ambient environment, heat exchangers, heat sinks, or areas internal to the body; humidity; activity, such as motion using a potentiometer type; location, such as those using Global Positioning System (GPS) data; user physiological data such as heart rate, pulse-oxygen or perspiration; remote sensing systems, such as cloud-based systems used for weather type data; air flow; and light.
6) Adjusting stored settings related to safety 168 such as alerts of the visual, audible or tactile type. In an embodiment, these alerts would be related to the native smart phone 161 speakers 174, vibratory mechanisms (not shown) and/or graphical user interface on said smart phone's screen 172.
7) Associating the app with systems for communication 169 for use with the apparatus 100, such as wireless networks including Bluetooth type. Communication of this type is advantageous because it is capable of linking the apparatus with other networked devices, such as those that are part of the “Internet of Things”, like room thermostat-type devices.
8) Changing settings for data management 170. These settings can include, for example: preferences for gathering input about the level of satisfaction with the thermal comfort being created by the apparatus 100; background transfer of information about the performance of the apparatus 100 such as temporal, temperature and power use data; and temporal frequency of data transfer.
9) Adjusting preference 171 settings such that the operation of the apparatus 100 is tailored to the needs of users, such as managing parameters and data related to: privacy; demographics; and geography.
10) In an embodiment, additional app 162a controls and features, such as the Thermostat interface 162c of FIG. 3C, are available by navigating using a hidden menu 173, such as a drop-down type.

With reference to FIG. 3C, in an embodiment, the software 160 can be further comprised of the following 15 numbered elements, as disclosed in the Thermostat interface 162c of the smartphone 161 app 162a.
1) A cog icon 175 enables user navigation to the Settings interface 162b of FIG. 3B.
2) A heat exchange type text element 176 indicates if the heat exchangers are providing a heating or cooling sensation.
3) An arcing slider element 177a indicates the adjustment level for the temperature of the heat exchangers.
4) The user can adjust the level of the temperature of the heat exchangers by moving a circular element 177b connected to the at the end point of the arcing slider element 177a.
5) The temperature of the heat exchangers in degrees is indicated using a surface temperature numeric element 178.
6) The ambient temperature in degrees is indicated using an ambient temperature numeric element 179a.
7) The ambient relative humidity percentage is indicated using an ambient humidity numeric element 179b.
8) The slider bar element 180a contains a cooling circular element and tooltip 180b for adjusting the thermostat ambient temperature setpoint above which the heat exchange should be of the cooling type.
9) The slider bar element 180a also contains a cooling circular element and tooltip 180c for adjusting the thermostat ambient temperature setpoint below which the heat exchange should be of the heating type.
10) A Modes icon 181 enables navigation to the Modes section described in FIG. 3A 165.
11) A Timer icon 182 enables the user to navigate to an interface (not shown) for adjusting the temporal period of operation for the system.
12) A Schedule icon 183 enables the user to navigate to an interface (not shown) for adjusting periods of operation for the system related to calendar days and hours of the day.
13) A power icon 184 enables the user to turn the system on and set it into a standby/off state.
14) A Bluetooth icon 185 indicates the connection status for the wireless connection to the system 100.
15) A battery icon 186 indicates the system 100 state of charge.

While this embodiment discloses the software 160 on a smart phone 161 app 162a, those skilled in the art will recognize that other related embodiments are possible, such as a desktop app, networked/client-server app, cloud app or any similar digital technology capable of controlling the hardware components and/or parameters stored in memory.

Those skilled in the art will also appreciate that control over the apparatus can be managed by parties other than the wearer, and that multiple examples of the apparatus can be managed from a central point of control. For example, a farmer could manage the comfort of a herd of cows wearing the apparatuses, and personnel at a stadium could manage embodiments embedded in spectator seating.

Operation of an Apparatus Embodiment

Use of the apparatus for delivering a sensation of cooling, in an embodiment, is done as described in the eight numbered paragraphs immediately below. While the use of the system for delivering a sensation of cooling in an embodiment is done as described in these paragraphs, those skilled in the art will appreciate that an embodiment and the description of its operation is only one of many possible solutions for the functions and operation of the disclosed apparatus. For example, the apparatus can include an electronic switch, such as a dual-pole dual-throw (DPDT) type, used to change the direction of the electric current through the thermoelectric module heat exchangers to alternate the direction of the heat exchange.

With reference to FIG. 2D, the following eight numbered sections represent a process for user operation, in an embodiment, of the apparatus 100:
1) The primary battery 114a is charged using a wall pack connected to an electrical outlet and connecting the power cable 113 to the power port 119. The system control 130 button LED and display 132 LCD provide the user with information about charging status. Details of the various options for status are described in the table in FIGS. 3A-3C.
2) The secondary battery 114b can be charged using a wall pack connected to an electrical outlet and connecting the power cable 113 to the charging port (not shown) of the secondary battery 114b.
3) As needed, the user can power the apparatus using the primary battery 114a internal to the DCU 112 or with the primary battery 114a as well as the secondary battery 114b connected via the power cable 113 to the power port 119.
4) The user activates the apparatus using the system control 130 button, which will illuminate the related LED. The user can press the system control 130 button again to adjust the output of the effectors 104a and to put the apparatus into a low-power standby mode.
5) The thermostat setpoint can be adjusted with user-operable controls for increasing 131a and decreasing 131b the setpoint. The setpoint will appear in the display 132. FIG. 4B describes the relationship between the user setting options and the thermostat.
6) The display 132 can also indicate safety information about the following: the status of the effectors 104a; temperature readings from the ambient environment as obtained by the temperature sensor 134; and internal fuses (not shown) for the DCU 112. FIG. 4C describes the relationship between the components of the apparatus, the conditions under which the apparatus would generate safety warnings and how the warnings would be generated—as well as the action intended by the user once the warning has been generated.
7) The effectors 104a operate in a pre-defined sequence according to the commands from algorithm running on the controller 114a. The controller 114a also obtains readings from the cutaneous temperature sensors 120a in order to execute closed-loop multi-input multi-output (“MIMO”) control in the algorithms implemented on the controller 114a. FIG. 5 describes a basic pre-defined sequence of operation for three heat exchangers at distinct physical locations on a body—HE1, HE2 and HE3. This sequence includes twelve (12) seconds of operation and three (3) seconds for a period of inactivity (also known as “drift”) to be applied to the effectors 104a.
8) Effectors 104a deliver sensible heat to the cutaneous layer of a body, thereby activating subcutaneous thermoreceptors. Details about specific outcomes related to thermoreceptor activation are disclosed in the list below, in items 4 through 8 and in the associated figures.

While this use of the apparatus represents the approach used with an embodiment, those skilled in the art will recognize that alternative approaches can be employed such as control through a wirelessly connected software application or a remote user-operable control pad.

Multiple-Input Multiple-Output (MIMO) Control Algorithm Drawings

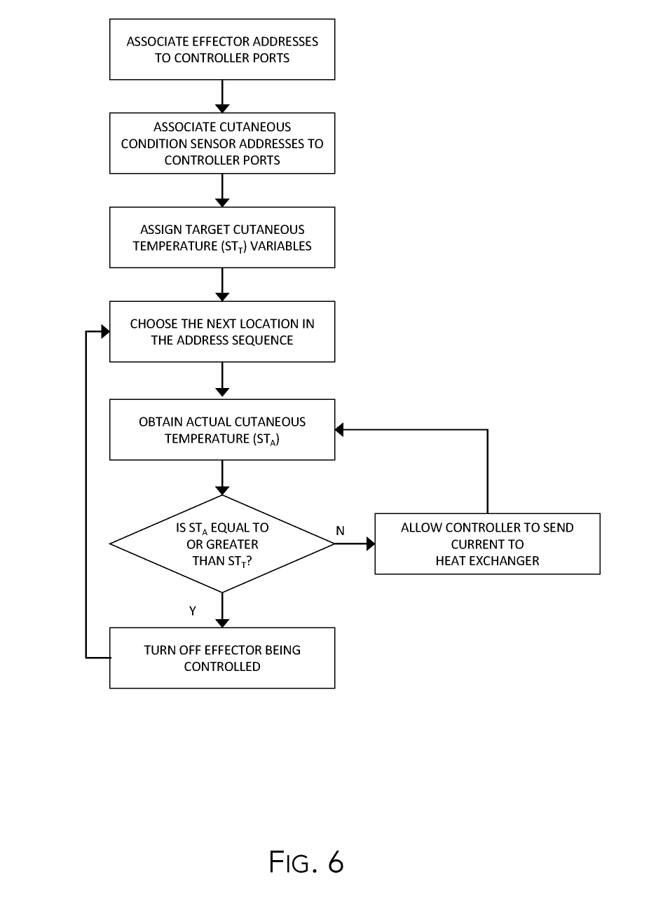

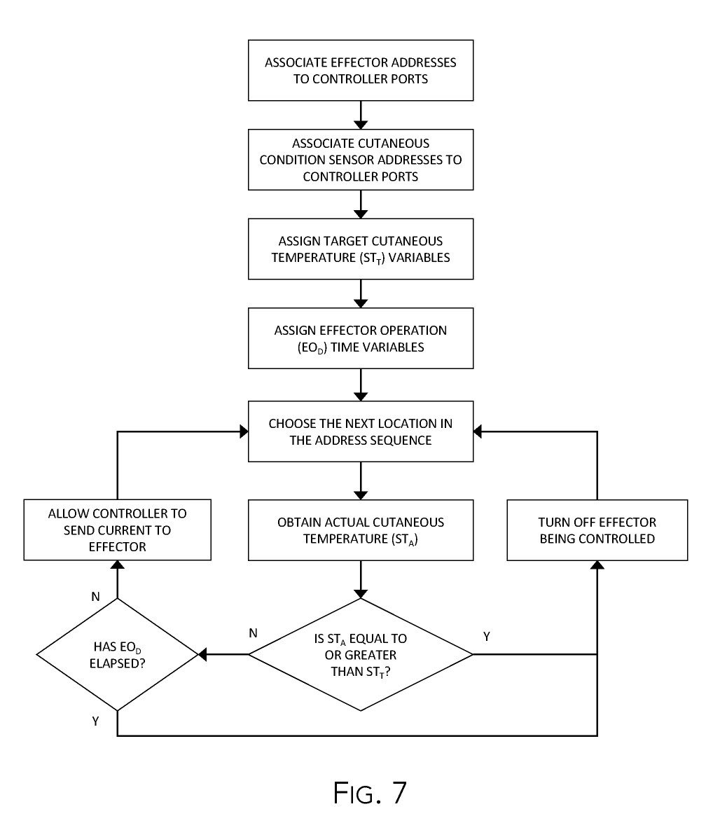

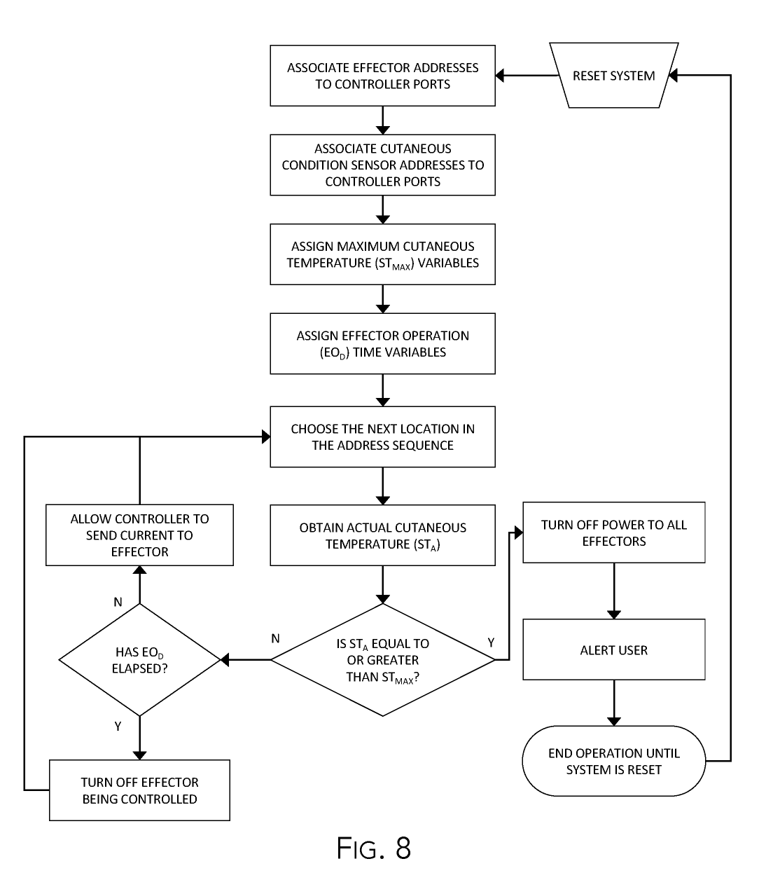

With reference to FIGS. 6-9B, Disclosed herein, four embodiments of basic multiple-input multiple-output (MIMO) control algorithms can include: baseline MIMO control; MIMO control with a time element; MIMO control for safety cutoff and MIMO control for energy efficient heat exchange.

With reference to FIG. 6, in an embodiment, the disclosed apparatus operates using a baseline MIMO control algorithm wherein: a sequence of effector location addresses are assigned to controller ports; a sequence of cutaneous condition sensor location addresses are assigned to controller ports; a target cutaneous temperature (ST_{T}) is assigned to effectors; an address in the sequence is chosen; the cutaneous temperature sensor at the chosen address is used to read an actual cutaneous temperature (ST_{A}); and, if ST_{A} is equal to or exceeds ST_{T}, the algorithm directs the controller to turn off the related effector. Operation continues sequentially using the remaining addresses in the sequence.

With reference to FIG. 7, in an embodiment, the disclosed apparatus operates using a time element MIMO control algorithm wherein: a sequence of effector location addresses are assigned to controller ports; a sequence of cutaneous condition sensor location addresses are assigned to controller ports; a target cutaneous temperature (ST_{T}) is assigned to effectors; a temporal reference for an effector total operating duration (EO_{D}) is assigned; an address in the sequence is chosen; the cutaneous temperature sensor at the chosen address is used to read an actual cutaneous temperature (ST_{A}); and, if ST_{A} is equal to or exceeds ST_{T}, and/or EO_{D} elapsed, the algorithm directs the controller to turn off the related effector. Operation continues sequentially using the remaining addresses in the sequence.

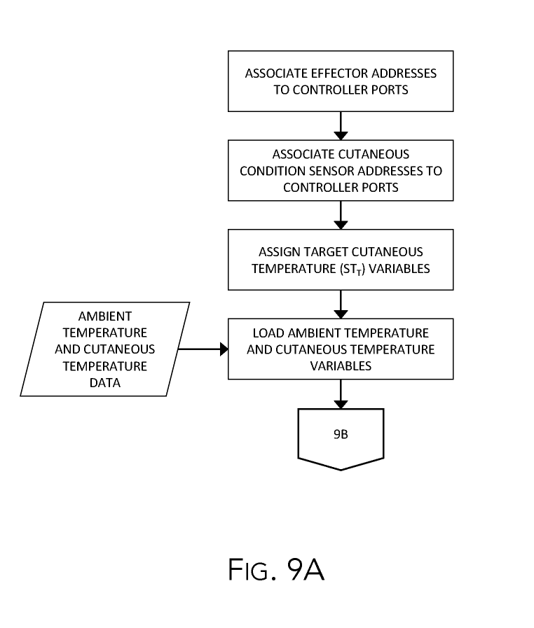

With reference to FIG. 8, in an embodiment, the disclosed apparatus operates using a safety cutoff MIMO control algorithm wherein: a sequence of effector location addresses are assigned to controller ports; a sequence of cutaneous condition sensor location addresses are assigned to controller ports; a maximum cutaneous temperature (ST_{T}) is assigned to effectors; a temporal reference for an effector total operating duration (EO_{D}) is assigned; an address in the sequence is chosen; the cutaneous temperature sensor at the chosen address is used to read an actual cutaneous temperature (ST_{A}); and, if ST_{A} is equal to or exceeds ST_{MAX}, power to all effectors is turned off, the user is alerted and the system must be rest. If ST_{MAX} is not reached but EO_{D} elapsed, the algorithm directs the controller to turn off the related effector and operation continues sequentially using the remaining addresses in the sequence. If ST_{MAX} is not reached and EO_{D} has not elapsed, the algorithm directs the controller to send current to the related effector and operation continues sequentially using the remaining addresses in the sequence.

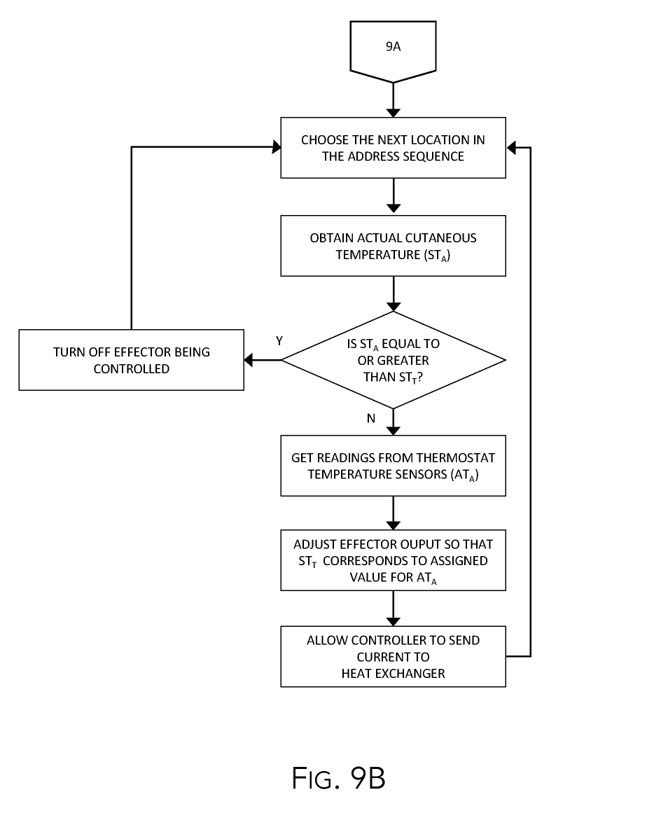

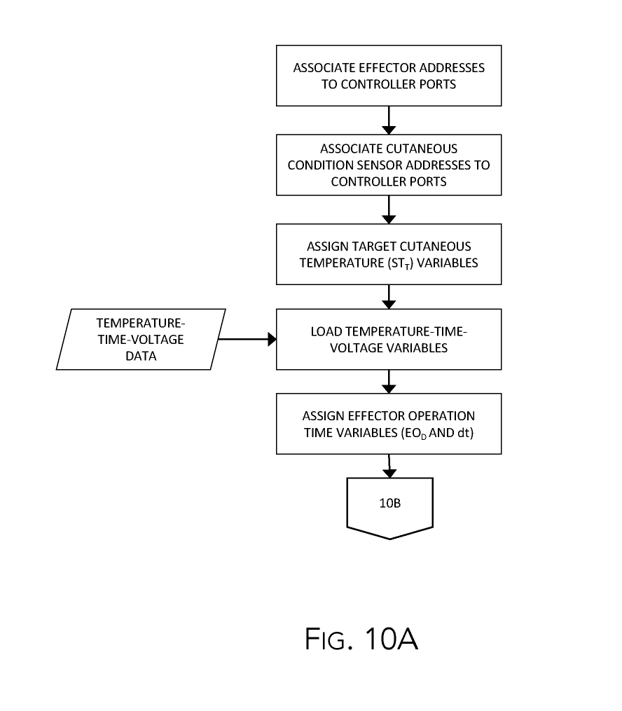

With reference to FIGS. 9A-9B, in an embodiment, the disclosed apparatus operates using a dynamic energy efficient MIMO control algorithm wherein: effector addresses are assigned to controller ports; cutaneous condition sensor addresses are assigned to controller ports; variables are assigned for target cutaneous temperatures (ST_{T}) at various ambient temperatures; an address in the sequence is chosen; cutaneous temperature sensors are used to read an actual cutaneous temperature (ST_{A}); ambient temperature sensors are used to read an actual ambient temperature (AT_{A}); and the algorithm directs the controller to operate power delivery to the related effector so that the value for ST_{T} at the specific corresponding value assigned for AT_{A} is maintained using readings of ST_{A} such that ST_{A} a does not exceed ST_{T}; and new value for ST_{T} is assigned when the value for AT_{A} changes. Operation continues sequentially using the remaining addresses in the sequence. This approach is advantageous because it does not use a single setpoint cutaneous temperature for a range of ambient temperatures, but instead dynamically adjusts the power delivery and cutaneous temperature to maintain comfort over a range of ambient temperatures. In an alternative embodiment, the apparatus would only operate within a defined range of ambient temperatures. In an alternative embodiment, sensors and the algorithm could measure and make use of ambient and cutaneous humidity readings in addition to ambient and cutaneous temperature readings.

Research-Driven Multiple-Input Multiple-Output (MIMO) Control Algorithm Drawings

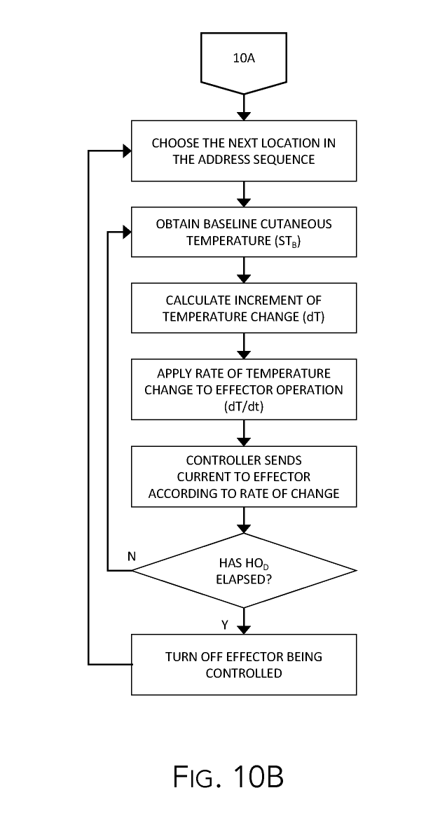

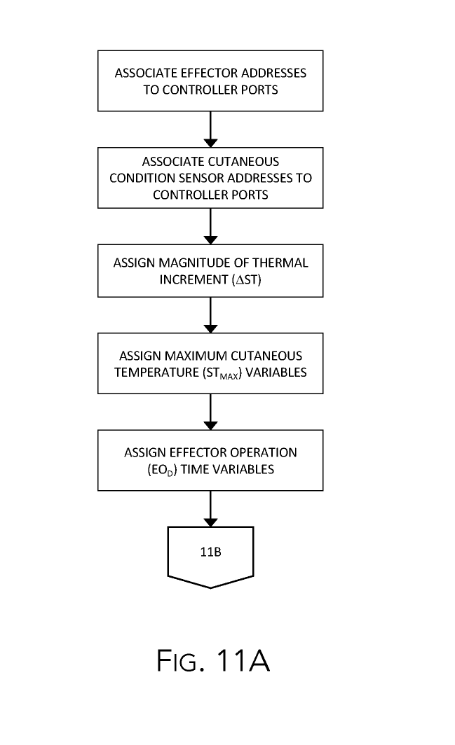

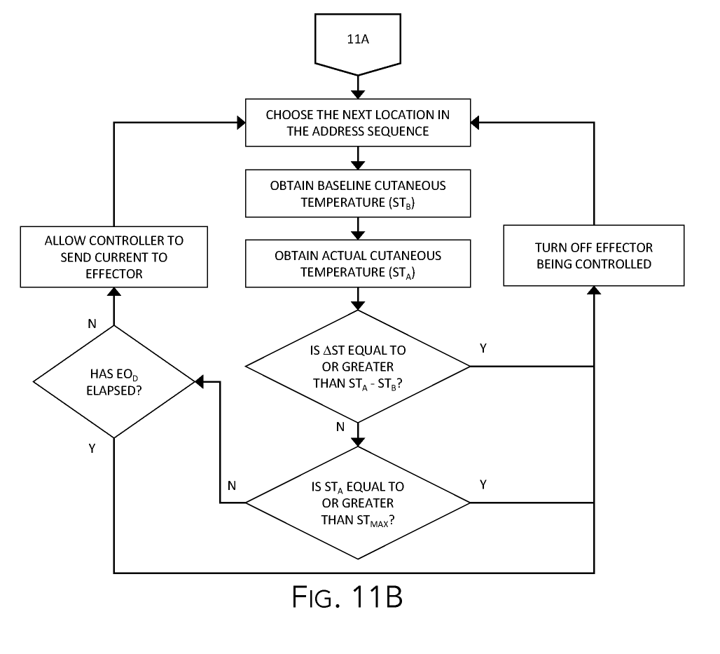

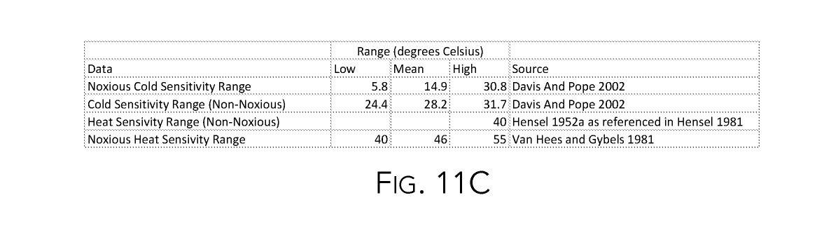

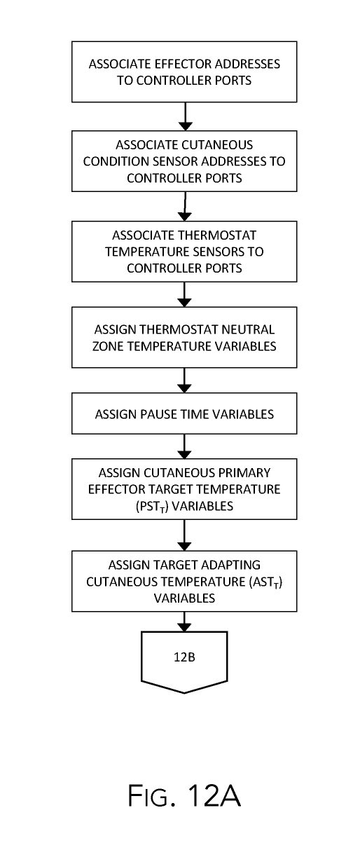

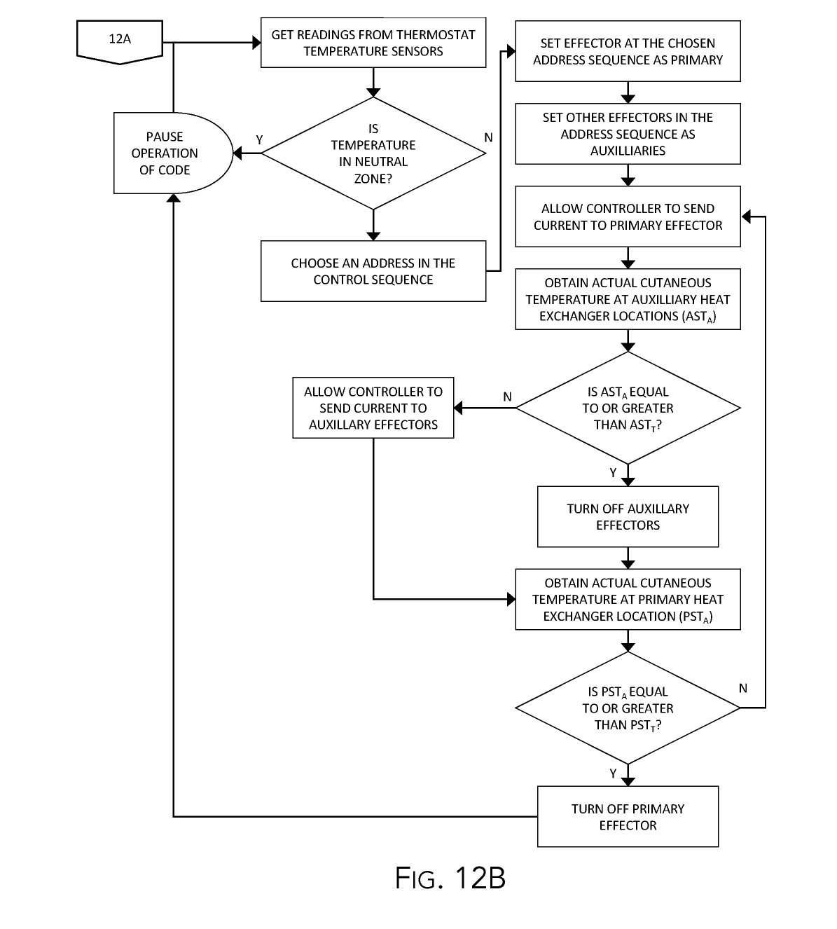

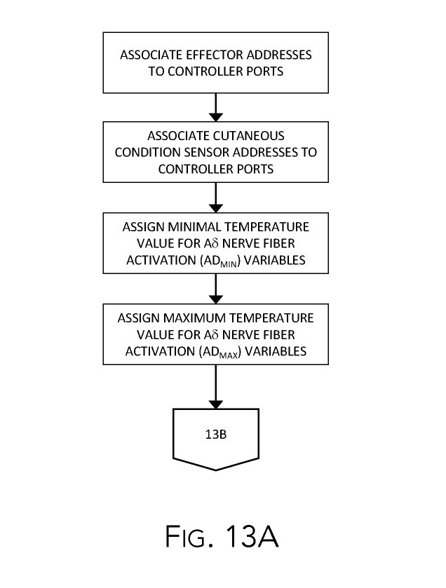

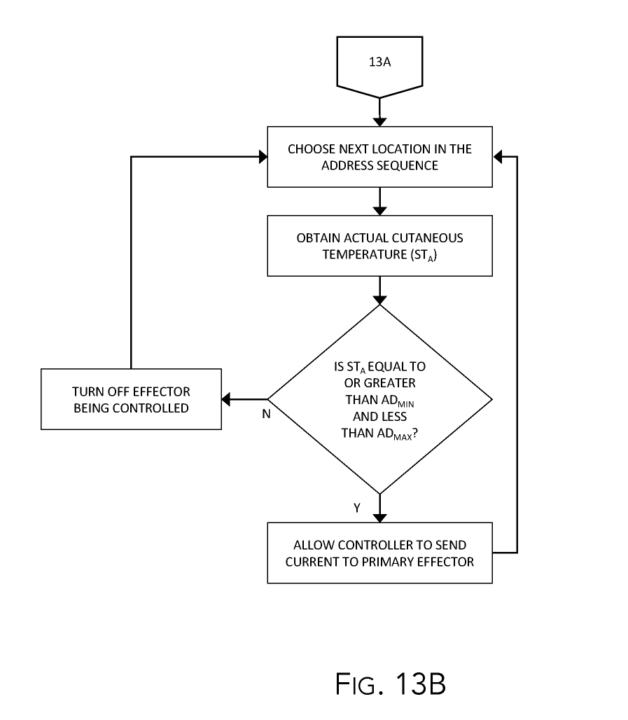

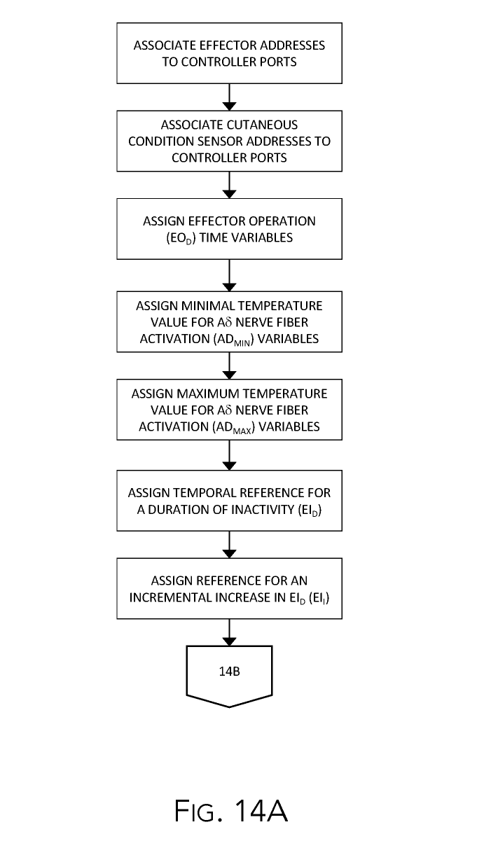

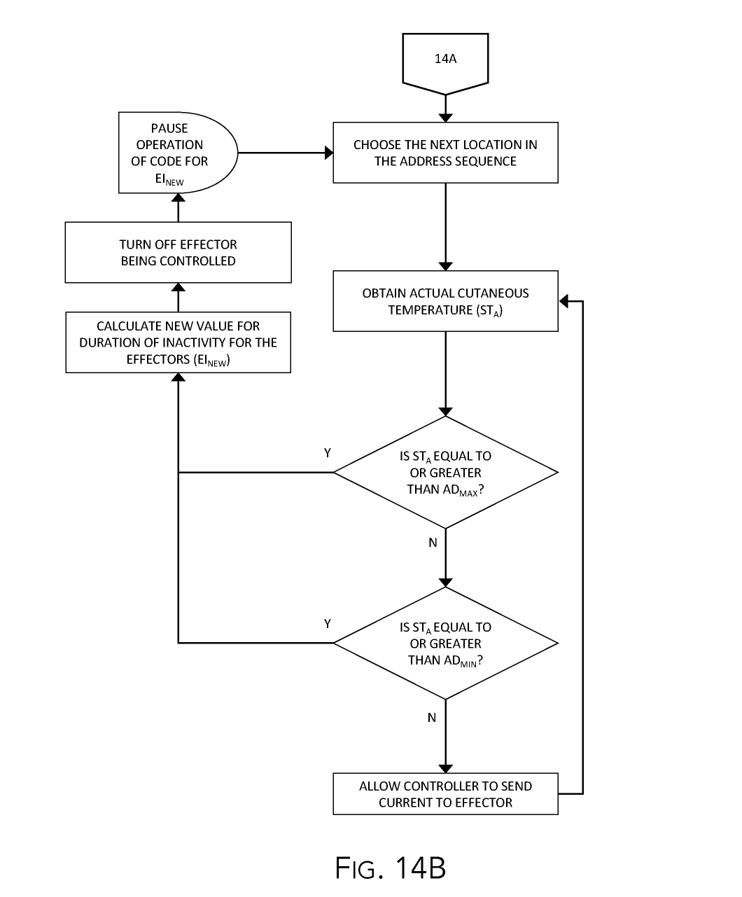

With reference to FIGS. 10A-14B, five research-driven algorithms for multiple-input multiple-output control strategies are disclosed in embodiments: FIGS. 10A-10B are flowcharts illustrating an embodiment where the disclosed apparatus utilizes a “rate of change” MIMO control strategy; FIGS. 11A-11B are flowcharts illustrating an embodiment where the disclosed apparatus utilizes a “thermal magnitude increment” MIMO control strategy; FIG. 11C is a specification table; FIGS. 12A-12B are flowcharts illustrating an embodiment where the disclosed apparatus utilizes an “adapting temperature” MIMO control strategy; FIGS. 13A-13B are flowcharts illustrating an embodiment where the disclosed apparatus utilizes a “gate control counterstimulation” MIMO control strategy; and FIGS. 14A-14B are flowcharts illustrating an embodiment where the disclosed apparatus utilizes an “attention diversion” MIMO control strategy.

With reference to FIGS. 10A-10B, in an embodiment, the disclosed apparatus operates using a rate of change algorithm wherein: effector addresses are assigned to controller ports; cutaneous condition sensor addresses are assigned to controller ports; pre-determined values for temperature change as a function of time at various voltages are assigned; a temporal reference for an effector operating duration (EO_{D}) is assigned; an address in the sequence is chosen; a baseline temperature (ST_{B}) is obtained by the cutaneous temperature sensors; a target cutaneous temperature (ST_{T}) is assigned to the effectors; a temporal reference for an increment of time (dt) is assigned; an increment of temperature change (dT) is calculated; temperature readings are obtained by the sensors; and the algorithm directs the controller to operate the effectors such that the voltage is limited using PWM to the value corresponding to dT during HO_{D} and the overall rate of change of temperature is limited to dT/dt. Operation continues sequentially using the remaining addresses in the sequence. This approach is advantageous because increasing the rate of temperature change results in a directly related increase in the average impulse frequencies of warm thermoreceptor units on human hairy skin, as shown in research from Hensel and Konietzny. In an alternative embodiment, activity at an effector address would stop if ST_{B} was equal to or greater than ST_{T}.

With reference to FIGS. 11A-11C, in an embodiment, the disclosed apparatus operates using an algorithm wherein: effector addresses are assigned to controller ports; cutaneous condition sensor addresses are assigned to controller ports; a maximum increment of thermal magnitude (ΔST), being the total amount of possible temperature change for an effector at an address, is assigned; a maximum cutaneous temperature (ST_{MAX}) is assigned; a target effector operating duration (EO_{D}) is assigned; an address in the sequence is chosen; a cutaneous temperature sensors is used to obtain a cutaneous temperature baseline (ST_{B}); a cutaneous temperature sensor is used to obtain an actual cutaneous temperature (ST_{A}); and ST_{B} is subtracted from ST_{A} and if the resulting value is greater than or equal to ΔST, the related effector is turned off or, if not, the algorithm directs the controller to operate the effectors such that ΔST is delivered during EO_{D} and ST_{A} does not exceed ST_{MAX}. Operation continues sequentially using the remaining addresses in the sequence. This approach is advantageous because said heat exchangers will exchange heat differently given variables including: the temperature of the ambient air around the effectors; the rate of air movement around the effectors; and the layers of material between the skin-facing side of the effector and the skin itself. In an embodiment, ST_{A} is continuously kept above the mean threshold for noxious cold, 14.9° C. described in medical research (Davis and Pope 2002) by operating the effector at that address to maintain a minimum cutaneous temperature (ST_{MIN}). In an alternative embodiment, effectors operate such that ST_{A} is kept below the mean threshold for noxious heat, 46° C., described in medical research (Van Hees and Gybels 1981), which is assigned as the value for ST_{MAX}. The boundaries of noxious hot and cold sensations as described in medical research are summarized in the table in FIG. 11C.

With reference to FIGS. 12A-12B, in an embodiment, the disclosed apparatus operates using an adapting temperature algorithm wherein: effector addresses are assigned to controller ports; cutaneous condition sensor addresses are assigned to controller ports; thermostat sensors, for the purpose of measuring ambient temperature, are assigned to controller ports; variables for a neutral zone, which define the temperature conditions under which the apparatus will not operate, are assigned; variables for pause times, which define the temporal conditions under which the apparatus will not operate, are assigned; a target cutaneous temperature related to the primary effector (PST_{T}) is assigned; a target adapting cutaneous temperature related to the auxiliary effectors (AST_{T}) is assigned; the system operates only when readings from the thermostat temperature sensors show that the ambient temperature is not within the neutral zone; an address in the sequence is chosen; primary and auxiliary effectors are assigned based on the chosen address, wherein the primary is the effector at that address and the other effectors are the auxiliaries; cutaneous temperature sensors are used to obtain an actual cutaneous temperature for the primary effector (PST_{A}); cutaneous temperature sensors are used to obtain an actual cutaneous temperature for auxiliary effectors (AST_{A}); the algorithm directs the controller to operate the primary and auxiliary effectors such that the primary effector achieves PST_{T} while AST_{T} is maintained by the plurality of auxiliary heat exchangers, and when AST_{A} is equal to or greater than AST_{T}, the related effector is turned off and when PST_{A} is equal to or greater than PST_{T}, the related effector is turned off; and when all effectors are turned off, there is a pause in the code before obtaining new data from the thermostat temperature sensors. Operation continues sequentially using the remaining addresses in the sequence. This approach is advantageous because increasing the adapting temperature results in a directly related increase in the average impulse frequencies of warm thermoreceptor units on human hairy skin (Konietzny and Hensel 1977).

With reference to FIGS. 13A-13B, in an embodiment, the disclosed apparatus operates using a gate control counterstimulation algorithm wherein: effector addresses are assigned to controller ports; cutaneous condition sensor addresses are assigned to controller ports; a minimal temperature value for Aδ nerve fiber activation (AD_{MIN}) is assigned; a maximum temperature value for non-painful Aδ nerve fiber activation (AD_{MAX}) is assigned; readings from cutaneous temperature sensors are obtained to establish an actual temperature (ST_{A}); and said algorithm directs the controller to turn off the effectors or operate the effectors such that the cutaneous temperature is maintained between AD_{MIN} and AD_{MAX}. Operation continues sequentially using the remaining addresses in the sequence. This approach is advantageous because the dominant sensation of temperature reaching the cerebrum is the warming or cooling being generated by the apparatus, and not the ambient air. In an embodiment, the apparatus operates the effectors such that AD_{MIN} is continuously kept above the high threshold for noxious cold, 30.8° C., described in medical research (Davis and Pope 2002) and AD_{MAX} is kept below the low threshold for noxious heat, 40° C., described in medical research (Van Hees and Gybels 1981), or other values from the noxious sensitivity ranges in FIG. 11C.

With reference to FIGS. 14A-14B, in an embodiment, the disclosed apparatus operates using an attention diversion algorithm wherein: effector addresses are assigned to controller ports; cutaneous condition sensor addresses are assigned to controller ports; a minimal temperature value for Aδ nerve fiber activation (AD_{MIN}) is assigned; a maximum temperature value for non-painful Aδ nerve fiber activation (AD_{MAX}) is assigned; a temporal reference for a pause in operation for the effectors (EI_{D}) is assigned; a reference for an incremental increase in EI_{D} (EI_{I}) is assigned; readings from said cutaneous temperature sensors are obtained to establish an actual temperature (ST_{A}). Wherein and the algorithm directs the controller to operate the effectors such that the cutaneous temperature is maintained in a range between AD_{MIN} and AD_{MAX}; when the cutaneous temperature reading is not within this range, the algorithm calculates (EI_{D}+EI_{I}) to obtain a new value for duration of inactivity for the effectors (EI_{NEW}), EI_{NEW} is used in place of the pre-existing value for EI_{D}, the controller turns off the effector being controlled; and operation is paused for EI_{D}. In an embodiment, a user-operable reset control (not shown) is capable of resetting the apparatus so that EI_{NEW} is cleared from memory and returned to the default setting for EI_{D}. Operation continues sequentially using the remaining effectors in the matrix. This approach is advantageous because it increases the potential energy efficiency of the apparatus.

It will be appreciated that the above descriptions provide exemplary, non-limiting configurations. Although the present invention has been described with reference to exemplary embodiments, it is not limited thereto. Those skilled in the art will appreciate that numerous changes and modifications may be made to the embodiments of the invention and that such changes and modifications may be made without departing from the true spirit of the invention.

It is therefore intended that the embodiments and their descriptions be construed to cover all such equivalent variations as fall within the true spirit and scope of the invention. For example, the apparatus could: exchange data with remote resources about the performance and settings of a specific apparatus for the purpose of improving the user experience of all users; include calculations in the algorithms to account for specific heat and specific gravity of materials in the apparatus; be incorporated into an article used for the protection of the wearer, such as a hazardous materials suit, body armor or a plate carrier; and incorporate the apparatus into seating, such as seats for offices, arenas or vehicles.

1) A control system and method comprised of:
a) a controller;
b) electronic memory storage connected to said controller;
c) a plurality of cutaneous condition sensors connected to said controller for measuring one or more physical conditions related to the cutaneous layer of a body;
d) a physical means for attaching each one of said plurality of cutaneous condition sensors to one of a plurality of physical locations on said cutaneous layer of a body such that a specific one of said plurality of cutaneous condition sensors is placed in a unique physical location adjacent to said cutaneous layer of a body;
e) a means in code executable by said controller by which said controller is able to store in said electronic memory storage a plurality of address parameters for each one of a plurality of said physical locations; and
f) an electronic means connected to said controller by which the controller is able to associate each one of said plurality of address parameters with said cutaneous condition sensors for the purpose of controlling said cutaneous condition sensors.

2) The system and method of claim 1 further comprising one or more closed-loop multiple-input multiple output (MIMO) algorithms stored in said electronic memory storage and executable by said controller.

3) The system and method of claim 2 further comprising a plurality of electronic means connected to said controller in claim 1 capable of controlling output voltage for the purpose of varying electric current to a plurality of provided connected electronic components.

4) The system and method of claim 2 further comprising:
a) a plurality of electronic effector means connected to said controller in claim 1 capable of affecting one or more physical conditions related to said cutaneous layer of a body;
g) a physical means for attaching each one of said plurality of electronic effectors to one of a plurality of physical locations on said cutaneous layer of a body such that a specific one of said plurality of cutaneous condition sensors and a specific one of said plurality of electronic effectors share a unique common physical location adjacent to said cutaneous layer of a body; and
h) an electronic means connected to said controller in claim 1 by which said controller is able to associate said address parameters with said electronic effectors for the purpose of controlling said electronic effectors.

5) The system and method of claim 2 further comprised of a MIMO time element algorithm executable by said controller in claim 1.

6) The system and method of claim 2 further comprised of a MIMO dynamic energy efficient algorithm executable by said controller in claim 1.

7) The system and method of claim 2 further comprised of a MIMO rate of change algorithm executable by said controller in claim 1.

8) The system and method of claim 2 further comprised of a MIMO adapting temperature algorithm executable by said controller in claim 1.

9) The system and method of claim 2 further comprised of a MIMO thermal magnitude increment algorithm executable by said controller in claim 1.

10) The system and method of claim 2 further comprised of a MIMO gate control counterstimulation algorithm executable by said controller in claim 1.

11) The system and method of claim 2 further comprised of a MIMO attention diversion algorithm executable by said controller in claim 1.

12) The system and method of claim 2 further comprised of:
a) an electronic alert means connected to said controller in claim 1; and
b) a MIMO safety cutoff algorithm executable by said controller.

13) The system and method of claim 2 further comprised of:
a) An electronic means connected to said controller in claim 1 for collecting data on ambient environmental conditions; and
b) a MIMO thermostat driven algorithm executable by said controller.

14) The system and method of claim 3 further comprised of an electronic means connected to said controller in claim 1 for measuring the operating conditions of said electronic effectors in claim 3.

15) The system and method of claim 1 further comprised of an electronic means connected to said controller in claim 1 for visually displaying information to a user.

16) The system and method of claim 1 further comprised of a plurality of user-operable controls connected to a provided apparatus connected to a separate provided controller, and connected via wireless means to said controller in claim 1.

17) The system and method of claim 3 further comprised of an electronic means wherein pulse width modulation (PWM) is used by said controller in claim 1 or said separate controller for voltage control of said effector means.

18) The system and method of claim 1 further comprised of a plurality of adhesives used as the physical means for attaching each one of said plurality of electronic components to one of a plurality of said physical locations.

19) The system and method of claim 1 wherein the apparatus is further attached to an article worn on said body.
